(19) United States
(12) Patent Application Publication
(10) Pub. No.: US 2023/0404592 A1
(43) Pub. Date: Dec. 21, 2023

(54) IMPLANT WITH INTRASACCULAR AND INTRAVASCULAR PORTIONS AND RELATED TECHNOLOGY

(71) Applicant: Covidien LP, Mansfield, MA (US)

(72) Inventor: Ashok Nageswaran, Irvine, CA (US)

(21) Appl. No.: 17/804,053

(22) Filed: May 25, 2022

(51) Int. Cl. A61B 17/12

(52) U.S. Cl. CPC A61B 17/12177

(57) ABSTRACT

An implant in accordance with an embodiment of the present technology is configured for treating an aneurysm at a treatment location within a patient's vasculature at which first, second, and third blood vessels converge. The implant comprises an elongate body configured to be deployed partly within the first blood vessel, partly within the second blood vessel, and partly within an intervening portion of the vasculature adjacent to the aneurysm. The implant further comprises a bulbous body configured to be deployed within the aneurysm. The bulbous body is connected to the elongate body at an intermediate wall portion along a length of the elongate body. Opposing edges of the elongate body at the intermediate wall portion at least partially define an opening through which blood flows between the treatment location and the third blood vessel when the implant is deployed.

### TECHNICAL FIELD

This disclosure is related to devices for treating aneurysms.

### BACKGROUND

An aneurysm is an abnormal bulging or ballooning at a weakening in a wall of a blood vessel. Causes of aneurysms include disease, injury, and congenital abnormality. Although aneurysms can occur in many different parts of the body, the most common locations are the aorta and the cerebral vasculature. It is estimated that 2% or more of the worldwide population harbors an unruptured cerebral aneurysm. Many of these cerebral aneurysms eventually rupture leading to severe complications, such as subarachnoid hemorrhage. Unfortunately, the prognosis for subarachnoid hemorrhage is poor. Most patients with this condition either die or suffer from long-term cognitive impairment. The probability of death or disability from a ruptured aortic aneurysm can be even higher than from a ruptured cerebral aneurysm. Fortunately, treatments for unruptured aneurysms currently exist and continue to improve. Many of these treatments involve reducing blood flow within an aneurysm and thereby promoting thrombosis and embolization. Aneurysms treated in this manner are significantly less likely to rupture than untreated aneurysms. These treatments have the potential to save thousands of lives every year. Accordingly, there is an ongoing public health need for improvement of these treatments.

### SUMMARY

An implant in accordance with at least some embodiments of the present technology is suitable for treating an aneurysm at a treatment location within a patient's vasculature at which a first blood vessel, a second blood vessel, and a third blood vessel converge. The implant includes an elongate first body and a bulbous second body. A length of the first body extends between a first end of the first body and an opposite second end of the first body. The first body comprises a first wall portion proximate to the first end. The first wall portion comprises a concave first inner surface and a convex first outer surface opposite the first inner surface along a thickness of the first wall portion. The first body further comprises a second wall portion proximate to the second end. The second wall portion comprises a concave second inner surface and a convex second outer surface opposite the second inner surface along a thickness of the second wall portion. The first body further comprises an arced third wall portion between the first and second wall portions. The second body is connected to the first body via the third wall portion. A length of the second body extends laterally away from the first body. A width of the third wall portion extends between a first side of the third wall portion and an opposite second side of the third wall portion. The first body further comprises a first side edge portion proximate to the first side and a second side edge portion proximate to the second side. The second side edge portion is spaced apart from the first side edge portion to define an opening. The implant is transitionable between a deployed state and a delivery state. In the deployed state, the first wall portion engages a wall of the first blood vessel via the first outer surface, the second wall portion engages a wall of the second blood vessel via the second outer surface, the second body is disposed at least partially within the aneurysm, and the opening is located relative to the third blood vessel such that at least some blood flow between the treatment location and the third blood vessel is via the opening. In the delivery state, profiles of the first body and the second body across their respective lengths are more compact than in the deployed state.

A system in accordance with at least some embodiments of the present technology is suitable for treating an aneurysm at a treatment location within a patient's vasculature. The system comprises a elongate shaft defining an axial lumen and comprising a proximal end portion and a distal end portion opposite the proximal end portion along a length of the shaft. The shaft is configured to move the distal end portion intravascularly toward the treatment location. The system further comprises an implant in a low-profile delivery state within the lumen. The implant is configured to expand from the delivery state to a deployed state. The implant comprises an elongate first body and a second body connected to the first body. A length of the first body extends between a first end of the first body and an opposite second end of the first body. The first body comprises a first wall portion proximate to the first end and extending along a first part of the length of the first body, a second wall portion proximate to the second end and extending along a second part of the length of the first body, and a third wall portion extending along a third part of the length of the first body between the first and second parts of the length of the first body. The third wall portion is distal to the first and second wall portions along the length of the shaft. The second body is connected to the first body via the third wall portion and is distal to the first body along the length of the shaft. A distance along the length of the shaft between the second body and the first end is at least 3 millimeters shorter than a distance along the length of the shaft between the second body and the second end to facilitate sequential deployment of the first and second wall portions at the treatment location.

A method in accordance with at least some embodiments of the present technology is suitable for treating an aneurysm at a treatment location within a patient's vasculature at which a first blood vessel, a second blood vessel, and a third blood vessel converge. The method comprises locating a bulbous body of an implant within the aneurysm and locating an elongate body of the implant within the vasculature outside the aneurysm after locating the bulbous body. Locating the elongate body comprises engaging a wall of the first blood vessel via a convex first outer surface of a first wall portion of the elongate body. Locating the elongate body further comprises engaging a wall of the second blood vessel via a convex second outer surface of a second wall portion of the elongate body. Locating the elongate body further comprises engaging a wall of the vasculature between the first and second blood vessels via a convex third outer surface of a third wall portion of the elongate body. The third wall portion comprises a width extending between a first side of the third wall portion and an opposite second side of the third wall portion. The elongate body comprises a first side edge portion proximate to the first side and a second side edge portion proximate to the second side. The second side edge portion is spaced apart from the first side edge portion to define an opening. Locating the elongate body further comprises locating the opening relative to the third blood vessel such that at least some blood flow between the treatment location and the third blood vessel is via the opening.

Examples of aspects of the present technology are described below as numbered clauses (1, 2, 3, etc.) for convenience. These are provided as examples and do not limit the present technology.

Clause 1. An implant for treating an aneurysm at a treatment location within a patient's vasculature at which a first blood vessel, a second blood vessel, and a third blood vessel converge, the implant comprising:
an elongate first body, wherein a length of the first body extends between a first end of the first body and an opposite second end of the first body, and wherein the first body comprises:
   a first wall portion proximate to the first end, the first wall portion comprising a concave first inner surface and a convex first outer surface opposite the first inner surface along a thickness of the first wall portion,
   a second wall portion proximate to the second end, the second wall portion comprising a concave second inner surface and a convex second outer surface opposite the second inner surface along a thickness of the second wall portion,
   an arced third wall portion between the first and second wall portions, wherein a width of the third wall portion extends between a first side of the third wall portion and an opposite second side of the third wall portion,
   a first side edge portion proximate to the first side, and
   a second side edge portion proximate to the second side, wherein the second side edge portion is spaced apart from the first side edge portion to define an opening; and
a bulbous second body connected to the first body via the third wall portion, wherein a length of the second body extends laterally away from the first body, and
wherein the implant is transitionable between:
   a deployed state in which:
      the first wall portion engages a wall of the first blood vessel via the first outer surface,
      the second wall portion engages a wall of the second blood vessel via the second outer surface,
      the second body is disposed at least partially within the aneurysm, and
      the opening is located relative to the third blood vessel such that at least some blood flow between the treatment location and the third blood vessel is via the opening, and
   a delivery state in which profiles of the first body and the second body across their respective lengths are more compact than in the deployed state.

Clause 2. The implant of clause 1, wherein the first body comprises:
an arced first end edge portion proximate to the first end; and
an arced second end edge portion proximate to the second end.

Clause 3. The implant of any one of the preceding clauses, wherein the first and second side edge portions extend continuously from the first end edge portion to the second end edge portion.

Clause 4. The implant of any one of the preceding clauses, wherein: the first body comprises:
an arced first end edge portion proximate to the first end, and
a looped second end edge portion proximate to the second end; and the first wall portion is tubular.

Clause 5. The implant of any one of the preceding clauses, wherein the first and second side edge portions extend continuously from the third wall portion to the first end edge portion.

Clause 6. The implant of any one of the preceding clauses, wherein:
the first body comprises:
   a looped first end edge portion proximate to the first end, and
   a looped second end edge portion proximate to the second end; and the first and second wall portions are tubular.

Clause 7. The implant of any one of the preceding clauses, wherein the implant is transitionable between the delivery state and an unconstrained state in which:
the second body extends away from the first body in a first direction;
the first wall portion extends away from the third wall portion in a second direction no more than 45 degrees offset from the first direction; and
the second wall portion extends away from the third wall portion in a third direction no more than 45 degrees offset from the first direction.

Clause 8. The implant of any one of the preceding clauses, wherein:
the third wall portion defines an arc along the width of the third wall portion between the first and second sides; and
an arc angle of the arc is no more than 180 degrees.

Clause 9. The implant of any one of the preceding clauses, wherein the second body comprises a concave inner surface and a convex outer surface opposite the inner surface of the second body along a thickness of the second body.

Clause 10. The implant of any one of the preceding clauses, wherein:
the implant defines an annular recess between the first and second bodies; and
the annular recess at least partially receives a neck of the aneurysm when the implant is in the deployed state.

Clause 11. The implant of any one of the preceding clauses, wherein:
the first and second bodies are connected to one another at a connection point along the length of the first body;
a first part of the length of the first body extends from the connection point to the first end;
a second part of the length of the first body extends from the connection point to the second end; and
the first part of the length of the first body is at least 30% shorter than the second part of the length of the first body to facilitate sequential deployment of the first and second wall portions at the treatment location.

Clause 12. The implant of any one of the preceding clauses, wherein:
the first and second bodies are connected to one another at a connection point along the length of the first body;
a first part of the length of the first body extends from the connection point to the first end;
a second part of the length of the first body extends from the connection point to the second end; and
the first part of the length of the first body is at least 3 millimeters shorter than the second part of the length of the first body to facilitate sequential deployment of the first and second wall portions at the treatment location.

Clause 13. The implant of any one of the preceding clauses, wherein:
the first, second, and third wall portions comprise a first mesh;
the second body comprises a second mesh; and
the implant further comprises a clamp at which the first and second meshes are connected to one another.

Clause 14. The implant of any one of the preceding clauses, wherein:
the second body comprises a wall comprising a concave fourth inner surface and a convex fourth outer surface opposite the fourth inner surface along a thickness of the wall; and
the second body engages a wall of the aneurysm via the fourth outer surface when the implant is in the deployed state.

Clause 15. A system for treating an aneurysm at a treatment location within a patient's vasculature, the system comprising:
a elongate shaft defining an axial lumen and comprising a proximal end portion and a distal end portion opposite the proximal end portion along a length of the shaft, wherein the shaft is configured to move the distal end portion intravascularly toward the treatment location; and
an implant in a low-profile delivery state within the lumen, wherein the implant is configured to expand from the delivery state to a deployed state, and wherein the implant comprises:
   an elongate first body, wherein a length of the first body extends between a first end of the first body and an opposite second end of the first body, and wherein the first body comprises:
      a first wall portion proximate to the first end and extending along a first part of the length of the first body,
      a second wall portion proximate to the second end and extending along a second part of the length of the first body, and
      a third wall portion extending along a third part of the length of the first body between the first and second parts of the length of the first body, wherein the third wall portion is distal to the first and second wall portions along the length of the shaft, and
   a second body connected to the first body via the third wall portion, wherein the second body is distal to the first body along the length of the shaft,
wherein a distance along the length of the shaft between the second body and the first end is at least 3 millimeters shorter than a distance along the length of the shaft between the second body and the second end to facilitate sequential deployment of the first and second wall portions at the treatment location.

Clause 16. The system of clause 15, wherein the implant is transitionable between the delivery state and an unconstrained state in which:
the second body extends away from the first body in a first direction;
the first wall portion extends away from the third wall portion in a second direction no more than 45 degrees offset from the first direction; and
the second wall portion extends longitudinally away from the third wall portion in a third direction no more than 45 degrees offset from the first direction.

Clause 17. A method for treating an aneurysm at a treatment location within a patient's vasculature at which a first blood vessel, a second blood vessel, and a third blood vessel converge, the method comprising:
locating a bulbous body of an implant within the aneurysm; and
locating an elongate body of the implant within the vasculature outside the aneurysm after locating the bulbous body, wherein locating the elongate body comprises:
   engaging a wall of the first blood vessel via a convex first outer surface of a first wall portion of the elongate body,
   engaging a wall of the second blood vessel via a convex second outer surface of a second wall portion of the elongate body,
   engaging a wall of the vasculature between the first and second blood vessels via a convex third outer surface of a third wall portion of the elongate body, the third wall portion comprising a width extending between a first side of the third wall portion and an opposite second side of the third wall portion, wherein the elongate body comprises a first side edge portion proximate to the first side and a second side edge portion proximate to the second side, and wherein the second side edge portion is spaced apart from the first side edge portion to define an opening, and
   locating the opening relative to the third blood vessel such that at least some blood flow between the treatment location and the third blood vessel is via the opening.

Clause 18. The method of clause 17, further comprising moving the implant intravascularly toward the treatment location while the implant is in a low-profile delivery state within an axial lumen of an elongate shaft, wherein locating the bulbous and elongate bodies of the implant comprises causing relative movement between the implant and the shaft to transition the implant from the delivery state toward an expanded deployed state.

Clause 19. The method of any one of the preceding clauses, wherein moving the implant intravascularly toward the treatment location comprises moving the implant intravascularly toward the treatment location while the third wall portion is distal to the first and second wall portions along a length of the shaft.

Clause 20. The method of any one of the preceding clauses, wherein:
a length of the elongate body extends between a first end of the elongate body and an opposite second end of the elongate body; and
moving the implant intravascularly toward the treatment location comprises moving the implant intravascularly toward the treatment location while a distance along the length of the shaft between the bulbous body and the first end is at least 30% shorter than a distance along the length of the shaft between the bulbous body and the second end.

Clause 21. The method of any one of the preceding clauses, wherein locating the elongate body comprises at least partially receiving a neck of the aneurysm at an annular recess defined by the implant between the bulbous body and the elongate body.

### BRIEF DESCRIPTION OF THE DRAWINGS

Many aspects of the present technology can be better understood with reference to the following drawings. The relative dimensions in the drawings may be to scale with respect to some embodiments of the present technology. With respect to other embodiments, the drawings may not be to scale. The drawings may also be enlarged arbitrarily. For clarity, reference-number labels for analogous components or features may be omitted when the appropriate reference-number labels for such analogous components or features are clear in the context of the specification and all of the drawings considered together. Furthermore, the same reference numbers may be used to identify analogous components or features in multiple described embodiments.

FIG. 1 is a perspective view of an implant in accordance with at least some embodiments of the present technology.

FIG. 2 is an end profile view of the implant shown in FIG. 1.

FIG. 3 is a side profile view of the implant shown in FIG. 1.

FIG. 4 is a cross-sectional end profile view of the implant shown in FIG. 1 taken along the line 4-4 in FIG. 3.

FIGS. 5 and 6 are enlarged views of different respective portions of FIG. 4.

FIG. 7 is a cross-sectional end profile view of the implant shown in FIG. 1 taken along the line 7-7 in FIG. 3.

FIG. 8 is an anatomical view showing a treatment location within a patient's vasculature.

FIG. 9 is a side profile view of the implant shown in FIG. 1 in an unconstrained state.

FIG. 10 is a partially cross-sectional side profile view of a system in accordance with at least some embodiments of the present technology including a shaft and the implant shown in FIG. 1 in a low-profile delivery state within the shaft.

FIG. 11 is a partially cross-sectional side profile view of the implant shown in FIG. 1 in a deployed state at the treatment location shown in FIG. 8.

FIGS. 12-14 are partially cross-sectional side profile views of the system shown in FIG. 10 at different respective times during deployment of the implant shown in FIG. 1 at the treatment location shown in FIG. 8.

FIG. 15 is an enlarged cross-sectional view of a portion of FIG. 14.

FIGS. 16-20 are additional partially cross-sectional side profile views of the system shown in FIG. 10 at different respective times during deployment of the implant shown in FIG. 1 at the treatment location shown in FIG. 8.

FIGS. 21-23 are perspective views of implants in accordance with other embodiments of the present technology.

### DETAILED DESCRIPTION

Implants in accordance with at least some embodiments of the present technology facilitate treatment of aneurysms at vessel junctions (e.g., bifurcations and/or trifurcations). As discussed above, treatment of an aneurysm can involve reducing blood flow within the aneurysm and thereby promoting thrombosis and embolization. One approach to reducing blood flow within an aneurysm includes deploying an occlusive device (e.g., an expandable agglomeration of mesh or coil structures) within the aneurysm. Aneurysms at vessel junctions, however, typically have larger necks than other aneurysms. This can complicate stable positioning of occlusive devices within these aneurysms. Another approach includes deploying a flow-diverting device (e.g., a stent) within a blood vessel from which an aneurysm originates. Such a device, for example, can be positioned across the neck of an aneurysm and expanded into apposition with the vessel wall around the neck. With the device in place, blood flow into the aneurysm can be sufficiently reduced to cause desirable thrombosis and embolization within the aneurysm. Conventional flow-diverting devices are tubular and held in place by outward force on a vessel wall rather than by interaction with an aneurysm neck. At a vessel junction, however, a tubular device that extends across an aneurysm neck likely also extends across the flowpath of one or more vessels that converge at the junction. This can undesirably interfere with normal blood flow through the junction.

An implant in accordance with at least some embodiments of the present technology includes an intrasaccular anchoring and/or occlusive portion connected to an intravascular anchoring and/or flow-diverting portion. These portions surprisingly complement one another to facilitate treatment of aneurysms at vessel junctions. For example, the intravascular portion can include first and second wings that conformably engage first and second blood vessels, respectively, on opposite sides of an aneurysm at a vessel junction. Between the wings, the intravascular portion can include a bridging intermediate portion defining an opening through which blood from a third blood vessel at the junction enters or exits the junction with little or no obstruction. The intrasaccular portion can facilitate alignment of the opening with the third blood vessel. The wings can facilitate stable positioning of the intrasaccular portion thereby reducing or eliminating any risk of dislocation even when the aneurysm neck is large. In this or another manner, implants in accordance with at least some embodiments of the present technology facilitate treatment of aneurysms at vessel junctions. In addition or alternatively, implants and related devices, systems, and methods in accordance with embodiments of the present technology can at least partially address one or more other problems associated with conventional technologies whether or not such problems are stated herein.

Specific details of several embodiments of the present technology are disclosed herein with reference to FIGS. 1-23. It should be noted, in general, that other embodiments in addition to those disclosed herein are within the scope of the present technology. For example, embodiments of the present technology can have different configurations, components, and/or operations than those disclosed herein. Moreover, a person of ordinary skill in the art will understand that embodiments of the present technology can have configurations, components, and/or operations in addition to those disclosed herein and that these and other embodiments can be without configurations, components, and/or operations disclosed herein without deviating from the present technology.

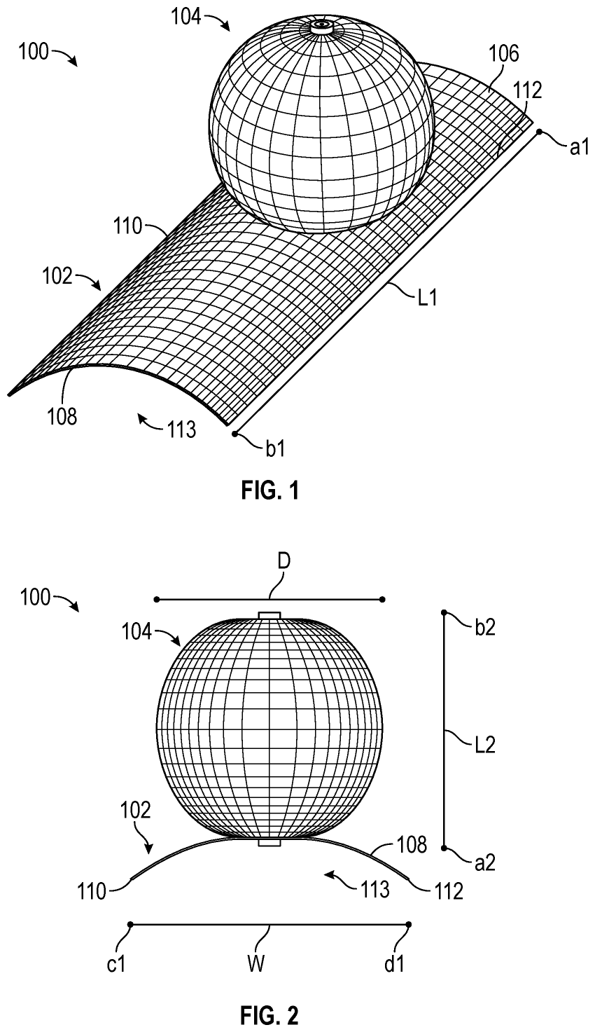

FIGS. 1 and 2 are a perspective view and an end profile view, respectively, of an implant 100 in accordance with at least some embodiments of the present technology. With reference to FIGS. 1 and 2 together, the implant 100 can include a first body 102 and a second body 104 operably connected to one another. The first body 102 can extend along a length L1 between a first end a1 and a second end b1 and along a width W between a first side c1 and a second side d1. The second body 104 can extend along a length L2 between a first end a2 and a second end b2. For example, the second body 104 can extend laterally away from the first body 102 along the length L2. The first and second bodies 102, 104 can be flexible or otherwise capable of transitioning between a variety of forms. In the form shown in FIGS. 1 and 2, the first body 102 is elongate and the second body 104 is bulbous and/or spheroid. The second body 104 can have a diameter D perpendicular to the length L2. The respective shapes of the first and second bodies 102, 104 can at least partially correspond to the shapes of anatomical features with which the first and second bodies 102, 104 interact when the implant 100 is deployed at a treatment location within a patient's vasculature. For example, the first body 102 can be shaped to conformably engage a wall of a blood vessel near an aneurysm and the second body 104 can be shaped to conformably engage a wall of the aneurysm. In at least some embodiments, the diameter D is within a range from 4 millimeters to 12 millimeters when the second body 104 is unconstrained. In these and other embodiments the width W can be within a range from 2 to 4 millimeters when the first body 102 is unconstrained. Furthermore, the width W can be smaller than the diameter D when the first and second bodies 102, 104 are unconstrained.

The first body 102 can include a first end edge portion 106 proximate to the first end a1 and a second end edge portion 108 proximate to the second end b1. In the illustrated embodiment, the first and second end edge portions 106, 108 are curved and/or arced and in respective planes perpendicular to the length L1. In other embodiments, counterparts of one or both of the first and second end edge portions 106, 108 can have different shapes, positions, and/or features. For example, counterparts of the first and second end edge portions 106, 108 can be straight, looped, and/or offset from respective planes perpendicular to the length L1. With reference again to FIGS. 1 and 2, the first body 102 can include a first side edge portion 110 proximate to the first side c1 and a second side edge portion 112 proximate to the second side d1. The first and second side edge portions 110, 112 can be spaced apart to define an opening 113. In the illustrated embodiment, the first and second side edge portions 110, 112 are straight and parallel to the length L1. The first and second side edge portions 110, 112 in the illustrated embodiment also extend continuously between the first and second end edge portions 106, 108. In other embodiments, counterparts of one or both of the first and second side edge portions 110, 112 can have different shapes, positions, and/or features. For example, counterparts of the first and second side edge portions 110, 112 can be curved, arced, and/or not parallel to the length L1. As another example, counterparts of the first and second side edge portions 110, 112 can extend along only a portion of the length L1, such as a portion spaced apart from one or both of the first and second ends a1, b1.

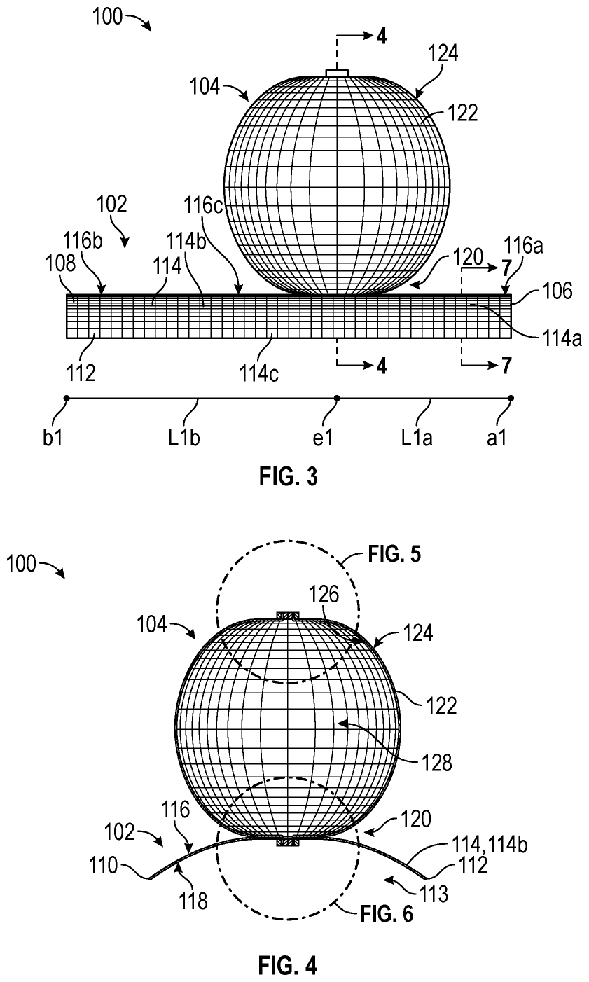

FIG. 3 is a side profile view of the implant 100. FIG. 4 is a cross-sectional end profile view of the implant 100 taken along the line 4-4 in FIG. 3. With reference to FIGS. 1-4 together, the first body 102 can be made up at least primarily of a wall 114 having an outer surface 116 and an inner surface 118 opposite to the outer surface 116 along a thickness of the wall 114. The thickness can be many times smaller than the length L1. For example, the thickness on average throughout the wall 114 can be no more than 0.5% of the length L1 (e.g., from 0.05% to 0.5% of the length L1 or from 0.05% to 0.3% of the length L1). In these and other cases, the thickness can be no more than 0.1 millimeter (e.g., within a range from 0.01 millimeter to 0.1 millimeter). These and other dimensional features of the first body 102 can be useful to reduce obstruction of blood flow, to increase flexibility, and/or for one or more other reasons.

As parts of the wall 114, the first body 102 can include a first wall portion 114a proximate to the first end a1, a second wall portion 114b proximate to the second end b1, and a third wall portion 114c therebetween. One, some, or all of the first, second, and third wall portions 114a-114c can be curved and/or arced along the width W. This can be useful, for example, to facilitate shape correspondence between the first, second, and third wall portions 114a-114c and one or more blood-vessel walls when the implant 100 is deployed at a treatment location. Relatedly, the first wall portion 114a can include a convex first outer surface 116a and a concave first inner surface (not shown) opposite the first outer surface 116a along a thickness of the first wall portion 114a. Similarly, the second wall portion 114b can include a convex second outer surface 116b and a concave second inner surface (not shown) opposite the second outer surface 116b along a thickness of the second wall portion 114b. Also similarly, the third wall portion 114c can include a convex third outer surface 116c and a concave third inner surface (not shown) opposite the third outer surface 116c along a thickness of the third wall portion 114c.

The implant 100 can define an annular recess 120 between the first and second bodies 102, 104. The second body 104 can be connected to the first body via the third wall portion 114c at a connection point e1 along the length L1. In at least some cases, the third wall portion 114c is centered at the connection point e1 and includes portions of the wall 114 of the first body 102 adjacent to the recess 120. The second body 104 can be radially symmetrical about an axis parallel to the length L2, perpendicular to the length L1, and passing through the connection point e1. As shown in FIG. 3, a first part L1a of the length L1 extending from the connection point e1 to the first end a1 can be shorter than a second part L1b of the length L1 extending from the connection point e1 to the second end b1. For example, the first part L1a of the length L1 can be shorter than the second part L1b of the length L1 by at least 20%, at least 30%, or at least 40%. In addition or alternatively, the first part L1a of the length L1 can be shorter than the second part L1b of the length L1 by at least 2 millimeters, at least 3 millimeters, or at least 4 millimeters. As further discussed below, this length difference can be useful, for example, to facilitate sequential deployment of the first and second wall portions 114a, 114b at a treatment location.

In the illustrated embodiment, the second body 104 comprises a wall 122 having a convex outer surface 124 and a concave inner surface 126 opposite the outer surface 124 along a thickness of the wall 122. The second body 104 can enclose a spheroid cavity 128 configured to be empty (as shown) and/or to be at least partially filled with an intrasaccular occlusive structure (e.g., an expandable agglomeration of mesh or coil structures) when the implant 100 is deployed at a treatment location. In other embodiments, the second body 104 can have another suitable form. For example, a counterpart of the second body 104 can include an intrasaccular occlusive structure that is not encased by a counterpart of the wall 122. Furthermore, counterparts of the second body 104 can have shapes other than spheroid. Relatedly, counterparts of the second body 104 can be configured to conformably engage some, none, or all of an aneurysm wall.

With reference again to FIGS. 1-4, the walls 114, 122 can include meshes of one or more interconnected wires woven together and/or formed together (e.g., 3D printed) in a network that enables the walls 114, 122 to expand and contract. Alternatively or in addition, the walls 114,122 can include perforated sheets. Furthermore, the walls 114,122 can be formed together or separately. In the latter case, the walls 114, 122 can be welded, soldered, glued, clamped, or otherwise connected to one another after being formed. Suitable materials for the walls 114, 122 include biocompatible metal alloys and polymers. For example, the walls 114,122 can include nitinol-platinum alloy drawn filled tube wires with average diameters of 20-30 microns. When formed from wires, the density of wires within the walls 114,122 can be selected to achieve a desired level of porosity and/or flexibility. In some embodiments, the wall 114 of the first body 102 includes between 96 and 144 individual wires. In these and other embodiment, the wall 122 of the second body 104 can include 12-36 wires. Other structural configurations, materials, and numbers of wires are also possible.

One or more features of the walls 114, 122 can vary throughout the implant 100 based on an intended position and/or function of a specific portion of the implant 100. For example, a portion of the wall 114 of the first body 102 configured to be positioned over the neck of an aneurysm (e.g., all or some of the third wall portion 114c) can have lower porosity and/or greater surface coverage than the wall 122 of the second body 104 or other portions of the wall 114 of the first body 102. This can be useful, for example, to promote thrombosis and embolization within an aneurysm without unduly reducing flexibility or other potentially desirable mechanical properties throughout the implant 100. In addition or alternatively, one or more portions of the implant 100 configured to anchor to a wall of a blood vessel (e.g., the first and second wall portions 114a, 114b) can have greater porosity, lower surface coverage, and/or be configured to exert greater chronic outward force than other portions of the implant 100.

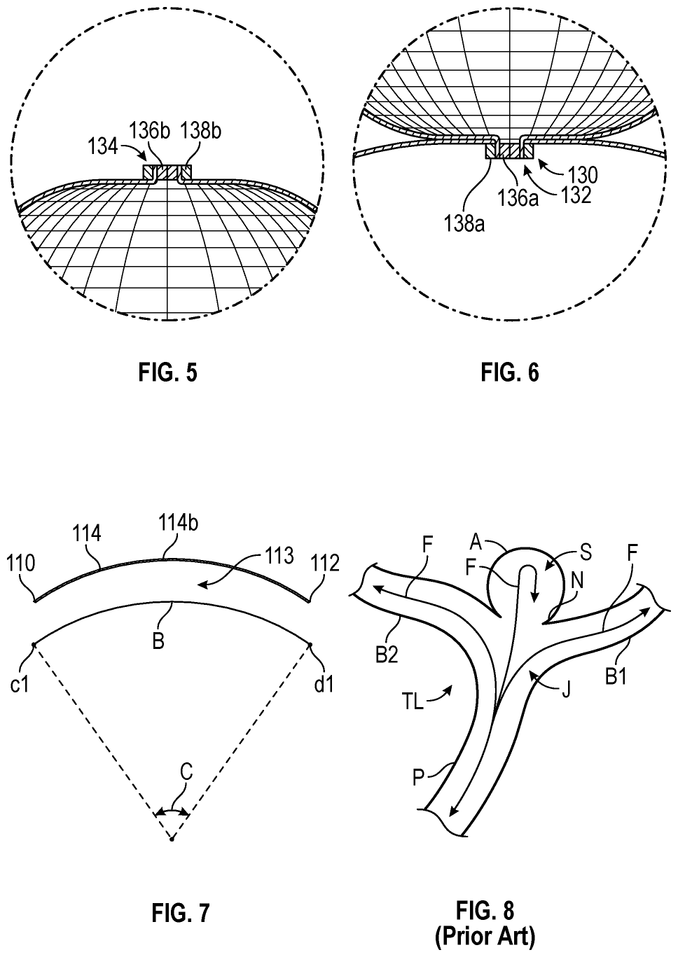

FIGS. 5 and 6 are enlarged views of different respective portions of FIG. 4. Specifically, FIG. 5 shows a portion of the second body 104 at the second end b2 of the length L2 and FIG. 6 shows a connection 130 between the first and second bodies 102, 104 at the first end a2 of the length L2. As shown in FIGS. 5 and 6, the implant 100 can include a first clamp 132 at the connection 130 and a second clamp 134 at the portion of the second body 104 at the second end b2 of the length L2. The first and second clamps 132, 134 can individually include a plug 136 (individually identified as plugs 136a, 136b) and a ring 138 (individually identified as rings 138a, 138b) extending around the corresponding plug 136a, 136b. With respect to the one or both of the first and second clamps 132, 134, the corresponding plug 136a, 136b and/or ring 138a, 138b can be more radiopaque than the walls 114, 122. In at least some embodiments, with respect to the one or both of the first and second clamps 132, 134, the corresponding plug 136a, 136b and ring 138a, 138b are coaxially nested radiopaque marker bands crimped together.

Forming the implant 100 can include creating a gap in a precursor of the first body 102 at the third wall portion 114c about midway along the width W. Forming the gap can include shifting wires at the third wall portion 114c such that the third wall portion 114c becomes resiliently biased toward closing the gap. The wall 122 of the second body 104 can be tubular before being connected to the wall 114 of the first body 102. In these and other cases, one end of the wall 122 of the second body 104 can be inserted through the gap in the wall 114 of the first body 102 and secured between the plug 136a and the ring 138a to form the first clamp 132. For example, the plug 136a, the ring 138a, and the end of the wall 122 of the second body 104 can be cinched against the wall 114 of the first body 102 and crimped together before or after the cinching. The plug 136a can be configured to collapse from an annular form to a non-annular form in response to the crimping. The second clamp 134 can be formed in a similar manner to close an opposite end of the wall 122 of the second body 104. By installing the first and second clamps 132, 134 or in another suitable manner, the wall 122 of the second body 104 can be changed from a tubular form to a bulbous and/or spheroid form.

FIG. 7 is a cross-sectional end profile view of the implant 100 taken along the line 7-7 in FIG. 3. With reference to FIGS. 1, 2 and 7 together, the wall 114 of the first body 102 can define an arc B along the width W between the first and second sides c1, d1. The arc B can have an arc angle C. In the illustrated embodiment, the opening 113, the arc B, and the arc angle C are consistent along the length L1. In other embodiments, a counterpart of the first body 102 can define an opening along only a portion of the length L1. In these and still other embodiments, the arc B can be absent or different in form and/or size at different portions of the length L1. For example, the first, second, and third wall portions 114a, 114b, 114c can individually define different respective arcs or no arc.

FIG. 8 is an anatomical view showing a treatment location TL within a patient's vasculature at which multiple blood vessels converge. For example, the treatment location TL can include a junction J at which a parent blood vessel P splits into two or more branch blood vessels B1, B2. An aneurysm A located between the branch blood vessels B1, B2 includes a generally spherical sac S and a neck N between the sac S and the junction J. Aspects of the present technology are further described below primarily in the context of the treatment location TL. It should be understood, however, that implants and related devices, systems, and methods in accordance with embodiments of the present technology can be used at a variety of other treatment locations. In another compatible treatment location, an aneurysm to be treated is between a parent blood vessel and a branch blood vessel rather than between two branch blood vessels. Furthermore, branch blood vessels at an alternative treatment location can be at substantially different angles, have substantially different sizes, and/or be present in a different quantity (e.g., three or more) relative to the branch blood vessels B1, B2. Furthermore, an aneurysm to be treated at an alternative treatment location can be offset with respect to a junction. For example, a neck of such an aneurysm can face toward and/or be open to a branch blood vessel rather than to a parent blood vessel and/or be tilted with respect to a plane created by blood vessels converging at a junction, such as into or out of the page when the junction is represented in the manner in which the junction J is represented in FIG. 8. Other variations relative to the treatment location TL are also possible.

The implant 100 and related devices, systems, and methods in accordance with embodiments of the present technology can be configured for treating aneurysms in any vasculature of a patient including, for example, a cerebral artery, a peripheral artery, a coronary artery, a pulmonary artery, an abdominal artery, a thoracic artery, an aortic artery, etc. As shown in FIG. 8, blood (represented by arrows F) flows from the parent vessel P into the branch vessels B1, B2 via the junction J at the treatment location TL. Blood also flows into the aneurysm A. This blood flow can cause the aneurysm A to rupture. Consequently, merely reducing or eliminating this blood flow into the aneurysm A, even without closure of the of the aneurysm, can reduce the risk of the aneurysm rupturing. As discussed above, however, reducing blood flow into the aneurysm A can also cause thrombosis, embolization, healing, and/or other forms of natural closure of the aneurysm A, leading to an even greater reduction in the risk of the aneurysm A rupturing.

With reference to FIGS. 1, 7 and 8 together, the first wall portion 114a can define an arc B sized to promote stable connection between the first wall portion 114a and the branch blood vessel B1. Similarly, the second wall portion 114b can define an arc B sized to promote stable connection between the second wall portion 114b and the branch blood vessel B2. In at least some embodiments, one or both of the first and second wall portions 114a, 114b define respective arcs B having arc angles C of about 180 degrees or otherwise within a range from 90 degrees to 270 degrees. The third wall portion 114c can define an arc B sufficiently small to reduce or eliminate obstruction of blood flow between the junction J and the parent blood vessel P. In at least some embodiments, the third wall portion 114c defines an arc B having an arc angle C of about 180 degrees or otherwise within a range from 60 degrees to 220 degrees.

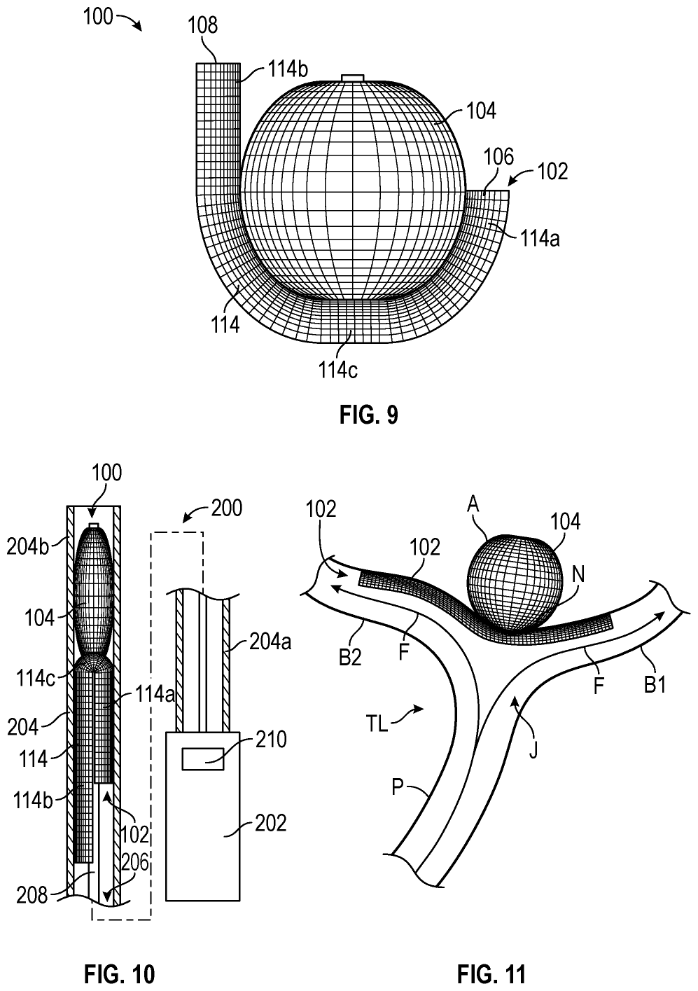

FIG. 9 is a side profile view of the implant 100 in an unconstrained state. With reference to FIGS. 3 and 8 together, the second body 104 can be bulbous and/or spheroid and the first body 102 can conformably extend around a portion of the second body 104 closest to the first body 102 such that the recess 120 is closed when the implant 100 is in the unconstrained state. In contrast to the state shown in FIG. 3, in the unconstrained state shown in FIG. 8, the length L1 can be curved, such as to form a U-shape and/or a J-shape. Furthermore, the first and second wall portions 114a, 114b can extend away from the third wall portion 114c in respective directions no more than 45 degrees offset from a direction in which the second body 104 extends away from the third wall portion 114c. These and/or other shape properties of the implant 100 in the unconstrained state can facilitate compatibility of the implant 100 with treatment locations of various sizes and configurations. For example, with reference to FIGS. 8 and 9 together, the first and second wall portions 114a, 114b can be configured to resiliently bend to accommodate various angles between the branch blood vessels B1, B2 or between one of the branch blood vessels B1, B2 and the parent blood vessel P when an aneurysm to be treated is located between one of the branch blood vessels B1, B2 and the parent blood vessel P. The shape of the implant 100 in the unconstrained state can be set thermally (e.g., by heat treating), structurally (e.g., by robotic wire bending), and/or in another suitable manner.

FIG. 10 is a partially cross-sectional side profile view of a system 200 in accordance with at least some embodiments of the present technology including the implant 100. The system 200 can include a handle 202 and an elongate shaft 204 connected to the handle 202. The shaft 204 can have a proximal end portion 204a closest to the handle 202 and a distal end portion 204b opposite the proximal end portion 204a along a length of the shaft 204. The shaft 204 can define an axial lumen 206 extending along the length of the shaft 204 between the proximal and distal end portions 204a, 204b. The system 200 can further include a mandrel 208 slidingly disposed within the lumen 206. At the handle 202 or another suitable location, the system 200 can include an actuator 210 operably connected to the mandrel 208 and/or to the shaft 204. For example, the actuator 210 can be configured to cause the mandrel 208 to move longitudinally within the lumen 206, to cause the shaft 204 to move longitudinally (e.g., to extend and/or to retract) relative to the mandrel 208, to cause a distal tip of the mandrel 208 to deflect laterally, to cause a distal tip of the shaft 204 to deflect laterally, and/or to otherwise manipulate the mandrel 208 and/or the shaft 204 during deployment of the implant 100 at a treatment location. The system 200 can include the implant 100 in a delivery state within the lumen 206, such as within a portion of the lumen 206 at the distal end portion 204b of the shaft 204. The shaft 204 can be configured to move the distal end portion 204b intravascularly toward a treatment location and thereby move the implant 100 to the treatment location. Alternatively or in addition, the implant 100 can be configured to move longitudinally through the lumen 206, such as along a guide wire (not shown).

FIG. 11 is a partially cross-sectional side profile view of the implant 100 in a deployed state at the treatment location TL. With reference to FIGS. 9-11 together, the implant 100 can be transitionable between the unconstrained state (FIG. 9), the delivery state (FIG. 10), and the deployed state (FIG. 11). The delivery state can be a low-profile state from which the implant 100 expands (e.g., self-expands and/or expands in response to an applied expansion force, such as from a balloon) to transition to the deployed state or the unconstrained state. For example, profiles of the first body 102 and the second body 104 across the lengths L1, L2, respectively, can be more compact when the implant 100 is in the delivery state than when the implant 100 is in the deployed state or the unconstrained state. As shown in FIG. 10, when the implant 100 is in the delivery state, the second body 104 can be distal to the first body 102. Furthermore, the third wall portion 114c can be curved such that the first and second wall portions 114a, 114b extend proximally from the third wall portion 114c. With reference to FIGS. 1 and 10 together, a first distance along the length of the shaft 204 between the second body 104 and the first end a1 of the length L1 of the first body 102 can be shorter than a second distance along the length of the shaft 204 between the second body 104 and the second end b1 of the length L1 of the first body 102. For example, the first distance can be shorter than the second distance by at least 20%, at least 30%, or at least 40%. In addition or alternatively, the first distance can be shorter than the second distance by at least 2 millimeters, at least 3 millimeters, or at least 4 millimeters.

With reference to FIGS. 1, 3 and 11 together, when the implant 100 is in the deployed state at the treatment location TL, the first wall portion 114a can conformably engage a wall of the branch blood vessel B1 via the first outer surface 116a. Similarly, the second wall portion 114b can conformably engage a wall of the branch blood vessel B2 via the second outer surface 116b. The third wall portion 114c can conformably engage a wall of the vasculature between the branch blood vessels B1, B2 via the third outer surface 116c. The second body 104 can be disposed at least partially within the aneurysm A and can engage a wall of the aneurysm A via the outer surface 124. The recess 120 can at least partially receive the neck N of the aneurysm A. The opening 113 can be located relative to the parent blood vessel P such that at least some blood flow from the parent blood vessel P into the treatment location TL is via the opening 113. Similarly, when the implant 100 is deployed at an alternative treatment location in which an aneurysm is located between a parent blood vessel and a first branch blood vessel, at least some blood flow from such a treatment location to a second branch blood vessel at the treatment location can be via the opening 113. As indicated by the arrows F in FIG. 11, deploying the implant 100 at the treatment location TL can at least partially reduce blood flow within the aneurysm A while having little or no effect on blood flow from the parent blood vessel P to the branch blood vessels B1, B2.

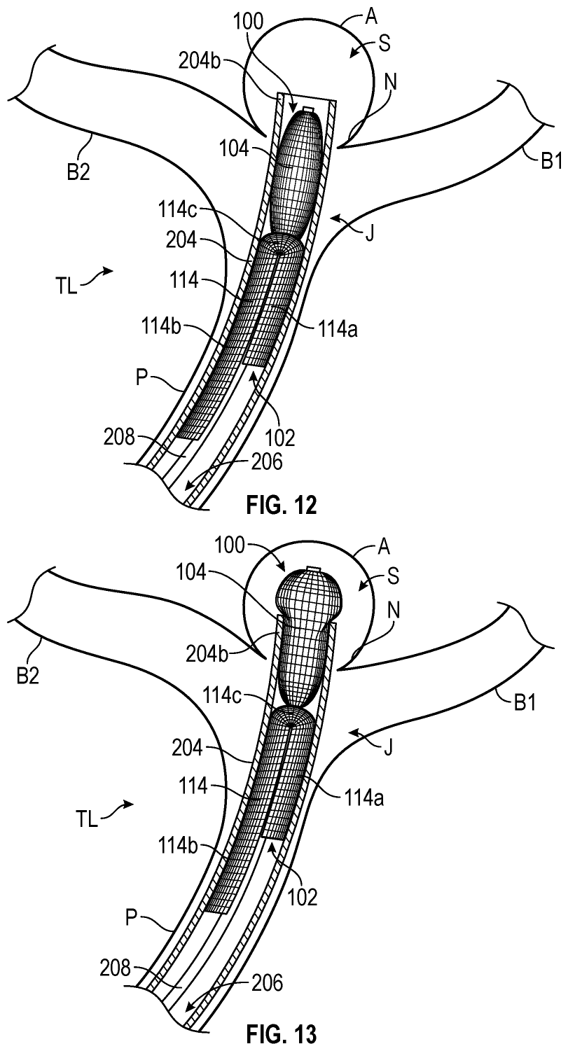

FIGS. 12-14 and 16-20 are partially cross-sectional side profile views of the system 200 at different respective times during deployment of the implant 100 at the treatment location TL. Deployment of the implant 100 can be part of a method for treating the aneurysm A at the treatment location TL in accordance with at least some embodiments of the present technology. For simplicity, aspects of the method are described primarily in the context of the system 200 and the implant 100. It should be understood, however, that the method, when suitable, and/or portions of the method, when suitable, can be practiced with respect to other systems and devices in accordance with embodiments of the present technology. As shown in FIG. 11, the method can include moving the implant 100 intravascularly toward the treatment location TL while the implant 100 is in the low-profile delivery state within the axial lumen 206 of the shaft 204. The method can further include causing relative movement between the implant 100 and the shaft 204. As shown in FIG. 12, this relative movement can transition the implant 100 from the delivery state toward the expanded deployed state at the treatment location TL.

A distal tip of the shaft 204 can be located within the aneurysm A before ejecting the implant 100 from the shaft 204. Alternatively, the distal tip of the shaft 204 can be located outside the aneurysm A before ejecting the implant 100 from the shaft 204. In either case, the method can include deploying the second body 104 within the aneurysm A after moving the implant 100 intravascularly toward the treatment location TL and before deploying the first body 102. Deploying the second body 104 within the aneurysm A can include moving the implant 100 distally relative to the shaft 204, such as by pushing the implant 100 via the mandrel 208. Alternatively or in addition, deploying the second body 104 within the aneurysm A can include retracting the shaft 204 proximally relative to the implant 100. As the second body 104 is released from the shaft 204, the second body 104 can self-expand toward its form when the implant 100 is in the deployed state. As discussed above, this form can be bulbous and/or spheroid. Moreover, an unconstrained diameter D of the second body 104 can be slightly larger (e.g., from 10% to 40% larger) than an effective diameter of the aneurysm A such that the second body 104 exerts a small force radially outward against the wall of the aneurysm A. While deploying the second body 104 within the aneurysm A and/or at another time during the method, the first clamp 132 (FIG. 6) can be visualized using fluoroscopy, such as to align the connection point e1 (FIG. 3) with a center of the neck N of the aneurysm A. In addition or alternatively, the second clamp 134 (FIG. 5) can be visualized using fluoroscopy, such as to confirm that the second body 104 is properly deployed within the aneurysm A.

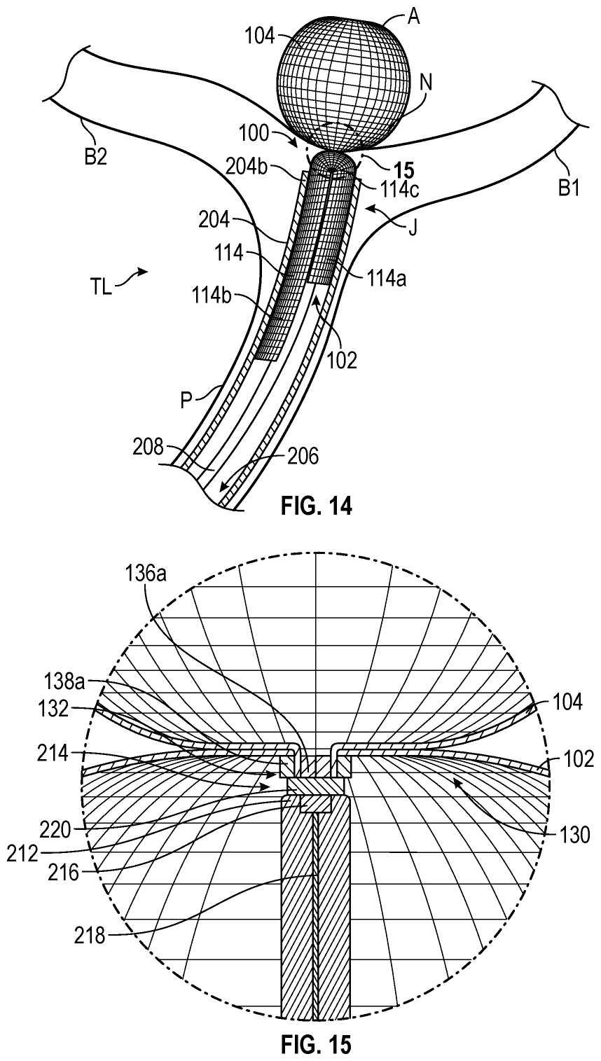

FIG. 15 is an enlarged cross-sectional view of a portion of FIG. 14. As shown in FIG. 15, the mandrel 208 can be detachably connected to the implant 100. For example, the mandrel 208 can include a distal tip 212 and a detachment mechanism 214 through which the mandrel 208 is detachably connected to the implant 100 via the first clamp 132. The detachment mechanism 214 can include a heater 216 at the distal tip 212 and an electrical conductor 218 extending proximally from the heater 216 to the handle 202 (Figure The detachment mechanism 214 can further include a volume of heat-sensitive adhesive 220 (e.g. solder) between the distal tip 212 and the first clamp 132. The handle 202 can include a power supply (not shown) and a switch (also not shown) that causes electricity to flow from the power supply to the heater 216 via the electrical conductor 218. This can cause the heater 216 to heat the adhesive 220 to a temperature sufficient to cause the adhesive 220 to reflow or otherwise reduce or eliminate adhesion between the distal tip 212 and the first clamp 132. Accordingly, the implant 100 can be released from the mandrel 208 at an appropriate time during deployment of the implant 100 at the treatment location TL, such as after the second body 104 is expanded within the aneurysm A as shown in FIG. 14. In other embodiments, a counterpart of the detachment mechanism 214 can be configured to detach the implant 100 electrolytically, mechanically, and/or chemically in addition to or instead of thermally. Furthermore, the detachment mechanism 214 can be eliminated. For example, the mandrel 208 can simply abut the first clamp 132 or another suitable portion of the implant 100.

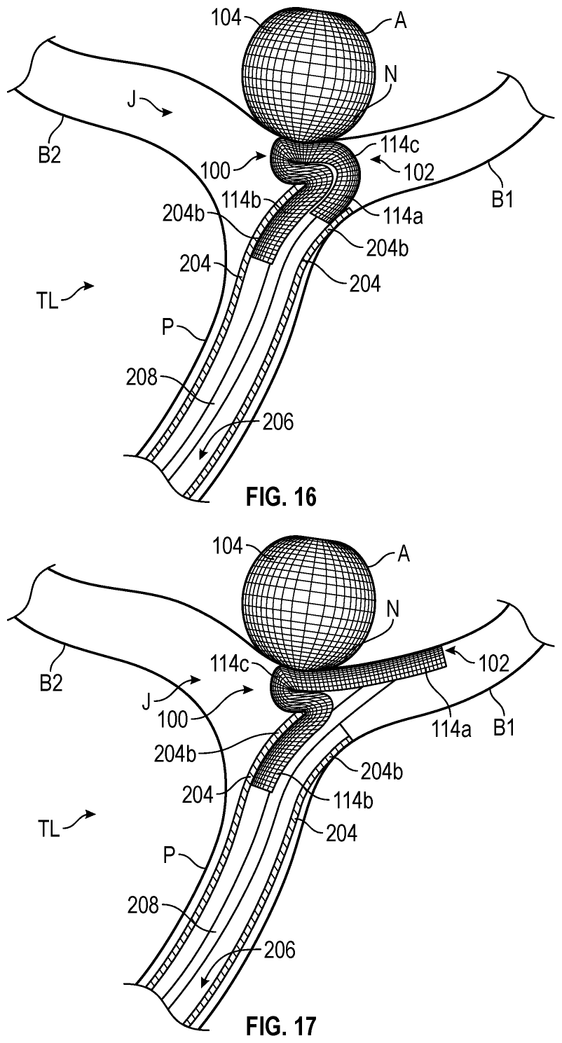

Deploying the implant 100 at the treatment location TL can further include deploying the first body 102 within the vasculature outside the aneurysm A after deploying the second body 104 within the aneurysm A. As shown in FIG. 16, after detaching the implant 100 from the mandrel 208, the shaft 204 can be repositioned toward the branch blood vessel B1. This can cause a some of the third wall portion 114c to conformably engage a wall of the vasculature between the branch blood vessels B1, B2 via the third outer surface 116c. The mandrel 208 can then be moved distally relative to the shaft 204 such that the distal tip 212 contacts the first inner surface of the first wall portion 114a. As shown in FIG. 17, the first wall portion 114a can then conformably engage a wall of the branch blood vessel B1 via the first outer surface 116a. This can occur in response to force from the mandrel 208 against the inner surface of the first wall portion 114a, in response to self-expansion of the first wall portion 114a in the absence and/or reduction of constraint from the shaft 204, and/or in another suitable manner.

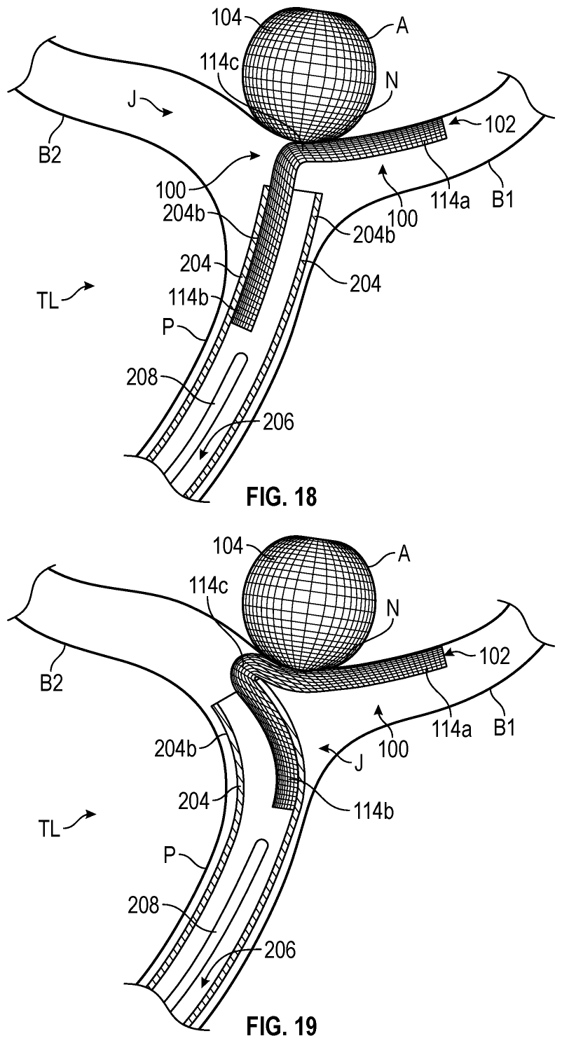

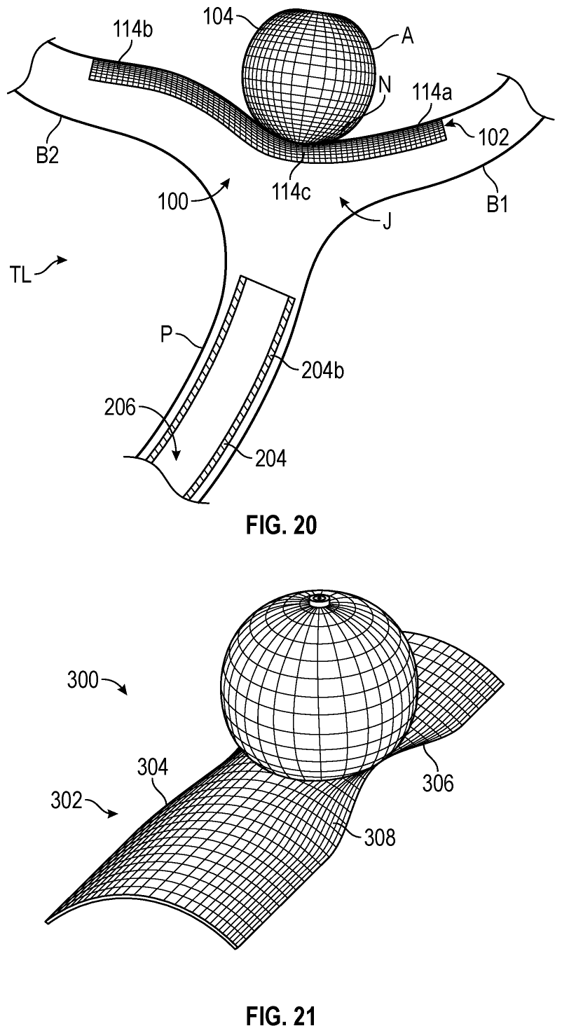

As shown in FIG. 18, once the first wall portion 114a is deployed, the mandrel 208 can be moved proximally relative to the shaft 204 away from the implant 100. As shown in FIG. 19, the shaft 204 can then be repositioned toward the branch blood vessel B2. This can cause a remainder of the third wall portion 114c to conformably engage the wall of the vasculature between the branch blood vessels B1, B2 via the third outer surface 116c. With reference to FIGS. 3 and 19 together, when the third wall portion 114c is deployed, the neck N of the aneurysm A can be at least partially received at the recess 120. From its position in FIG. 19, the shaft 204 can be moved distally into the branch blood vessel B2 to guide further deployment of the second wall portion 114b at the branch blood vessel B2. Once deployed, the second wall portion 114b can conformably engage a wall of the branch blood vessel B2 via the second outer surface 116b. The shaft 204 can then be withdrawn from the treatment location TL as shown in FIG. 20. With reference to FIGS. 3 and 20 together, the overall deployment of the first body 102 can locate the opening 113 relative to the parent blood vessel P such that at least some blood flow between the treatment location TL and the parent blood vessel P is via the opening 113.

In the method illustrated in FIGS. 12-20, both the shaft 204 and the mandrel 208 are manipulated to guide deployment of the first body 102 at the treatment location TL. In other embodiments, deployment of the first body 102 at the treatment location TL can involve only one or neither of these processes. For example, in some embodiments, the mandrel 208 is stowed proximally relative to the shaft 204 after detachment from the implant 100 rather than being used to guide deployment of the first body 102 at the treatment location TL. In other embodiments, the mandrel 208 can be used to guide deployment of the first body 102 at the treatment location TL without directing the shaft 204 toward the branch blood vessels B1, B2. With reference to FIGS. 3 and 12-20 together, the difference between the first and second parts L1a, L1b of the length L1 can facilitate sequential deployment of the first and second wall portions 114a, 114b at the treatment location TL. For example, even in the absence of the mandrel 208 as a manipulator, proximal movement of the shaft 204 relative to the implant 100 (e.g., while the implant 100 is anchored by the neck N of the aneurysm A) can cause the first wall portion 114a to be released from shaft 204 before the second wall portion 114b is released from the shaft 204. This sequential rather than simultaneous release of the first and second wall portions 114a, 114b from the shaft 204 can be useful, for example, to allow a clinician to better control alignment of the first and second wall portions 114a, 114b with the branch blood vessels B1, B2, respectively. This alignment can be controlled, for example, via rotation of the shaft 204 about an axis parallel to the length of the shaft 204. In addition or alternatively, sequential rather than simultaneous release of the first and second wall portions 114a, 114b from the shaft 204 can be useful to facilitate prepositioning the first and second wall portions 114a, 114b in the branch blood vessels B1, B2, respectively or otherwise guiding deployment of the first and second wall portions 114a, 114b through manipulation of the mandrel 208 and/or the shaft 204

FIG. 21 is a perspective view of an implant 300 in accordance with another embodiment of the present technology. The implant 300 can include features similar to or the same as the features described above with respect to the implant 100 shown in FIGS. 1-9. With reference to FIGS. 1-9 and 21 together, unlike the implant 100, the implant 300 includes an elongate body 302 extending along a width W that is different at different portions of the length L1. For example, the body 302 can include opposing first and second side edge portions 304, 306 that curve inwardly at an intermediate wall portion 308 centered at the connection point e1. The intermediate wall portion 308 can be spaced apart from the first and second ends a1, b1. For example, the intermediate wall portion 308 of the implant 300 can have the same position along the length L1 as the third wall portion 114c of the implant 100 has along the length L1. In at least some cases, the intermediate wall portion 308 has lower porosity than other portions of the body 302.

Forming the implant 300 can include the operations described above for forming the implant 100 followed by deforming the intermediate wall portion 308. This deforming can include reducing interstitial spaces between wires at the intermediate wall portion 308, such as by tightening a weave of the wires at the intermediate wall portion 308. This can both reduce the width W of the intermediate wall portion 308 and lower the porosity of the intermediate wall portion 308. Reduced width W of the intermediate wall portion 308 achieved in the described manner or another suitable manner can be useful to reduce the possibility of the intermediate wall portion 308 obstructing blood flow into or out of a blood vessel oriented toward an aneurysm proximate to the intermediate wall portion 308 when the implant is deployed at a treatment location. For example, with reference to FIGS. 8 and 21 together, the reduced width W of the intermediate wall portion 308 can reduce the possibility of the intermediate wall portion 308 obstructing blood flow between the junction J and the parent blood vessel P when the implant 300 is deployed at the treatment location TL. Lower porosity of the intermediate wall portion 308 achieved in the described manner or another suitable manner can be useful to reduce blood flow into the aneurysm A when the implant 300 is deployed at the treatment location TL.

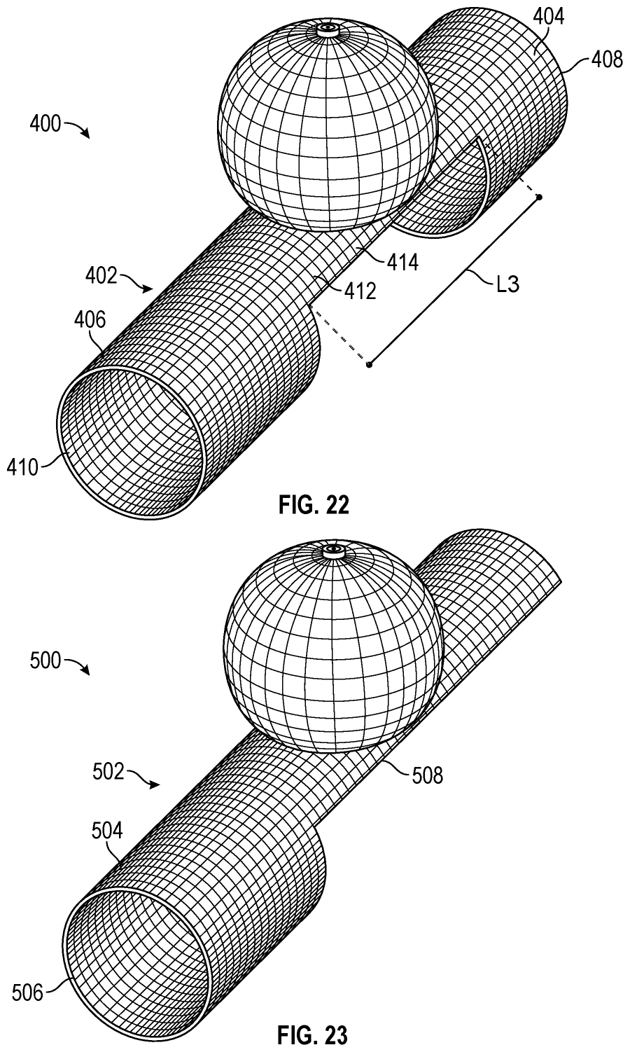

FIG. 22 is a perspective view of an implant 400 in accordance with another embodiment of the present technology. The implant 400 can include features similar to or the same as the features described above with respect to the implant 100 shown in FIGS. 1-9. With reference to FIGS. 1-9 and 22 together, unlike the implant 100, the implant 400 has an elongate body 402 including a tubular first wall portion 404 proximate to the first end a1 and a tubular second wall portion 406 proximate to the second end b1. Relatedly, the implant 400 can have a looped first end edge portion 408 proximate to the first end a1 and a looped second end edge portion 410 proximate to the second end b1. The first and second wall portions 404, 406 and the first and second end edge portions 408, 410 of the implant 400 can have the same respective positions along the length L1 as the first and second wall portions 114a, 114b and the first and second end edge portions 106, 108 of the implant 100 have along the length L1. The implant 400 can further include a third wall portion 412 having the same position along the length L1 as the third wall portion 114c of the implant 100 has along the length L1. The implant 400 can also include a first side edge portion 414 and an opposing second side edge portion (not shown) similar to the first and second side edge portions 110, 112 of the implant 100, but terminating at the first and second wall portions 404, 406 rather than at the first and second ends a1, b1. With reference to FIGS. 2, 8 and 22 together, in at least some cases, a distance L3 between the first and second wall portions 404, 406 is equal to or greater than the diameter D, within a range from 4 millimeters to 12 millimeters, and/or at least about twice a diameter of the neck N of the aneurysm A.

FIG. 23 is a perspective view of an implant 500 in accordance with another embodiment of the present technology. The implant 500 can include features similar to or the same as the features described above with respect to the implant 100 shown in FIGS. 1-9. With reference to FIGS. 1-9 and 23 together, unlike the implant 100, the implant 500 has an elongate body 502 including a tubular wall portion 504 proximate to the second end b1. Relatedly, the implant 500 can have a looped end edge portion 506 proximate to the second end b1. The wall portion 504 and the end edge portions 506 of the implant 500 can have the same respective positions along the length L1 as the second wall portion 114b and the second end edge portion 108 of the implant 100 have along the length L1. The implant 500 can also include a first side edge portion 508 and an opposing second side edge portion (not shown) similar to the first and second side edge portions 110, 112 of the implant 100, but terminating at the wall portion 504 and the first end a1 rather than at the first and second ends a1, b1.

With reference to FIG. 3, in yet another embodiment, a counterpart of the implant 100 includes a tubular wall portion and a looped end edge portion in place of the arced first wall portion 114a and the arced first end edge portion 106. Having a counterpart of the first wall portion 114a be tubular rather than arced can be useful, for example, to reduce or eliminate the possibility of the first wall portion 114a moving out of engagement with a wall of a blood vessel and potentially interfering with blood flow through the blood vessel. Similarly, having a counterpart of the second wall portion 114b be tubular rather than arced (e.g., as shown in FIG. 22) can be useful, for example, to reduce or eliminate the possibility of the second wall portion 114a moving out of engagement with a wall of a blood vessel and potentially interfering with blood flow through the blood vessel. Tubular rather than arcuate wall configurations also may provide stronger anchoring for the second body 104 and/or have other advantages. Arcuate rather than tubular wall configurations may provide advantages such as greater flexibility and lower delivery profile. Accordingly, both arcuate and tubular wall configurations can be useful in implants according to various embodiments of the present technology.

This disclosure is not intended to be exhaustive or to limit the present technology to the precise forms disclosed herein. Although specific embodiments are disclosed herein for illustrative purposes, various equivalent modifications are possible without deviating from the present technology, as those of ordinary skill in the relevant art will recognize. In some cases, well-known structures and functions have not been shown or described in detail to avoid unnecessarily obscuring the description of the embodiments of the present technology. Although steps of methods may be presented herein in a particular order, in alternative embodiments the steps may have another suitable order. Similarly, certain aspects of the present technology disclosed in the context of particular embodiments can be combined or eliminated in other embodiments. Furthermore, while advantages associated with certain embodiments may be disclosed herein in the context of those embodiments, other embodiments may also exhibit such advantages, and not all embodiments need necessarily exhibit such advantages or other advantages disclosed herein to fall within the scope of the present technology. This disclosure and the associated technology can encompass other embodiments not expressly shown or described herein.

Throughout this disclosure, the singular terms “a,” “an,” and “the” include plural referents unless the context clearly indicates otherwise. Similarly, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “comprising,” “including,” and the like are used throughout this disclosure to mean including at least the recited feature(s) such that any greater number of the same feature(s) and/or one or more additional types of features are not precluded. Directional terms, such as “upper,” “lower,” “front,” “back,” “vertical,” and “horizontal,” may be used herein to express and clarify the relationship between various structures. It should be understood that such terms do not denote absolute orientation. Furthermore, reference herein to “one embodiment,” “an embodiment,” or similar phrases means that a particular feature, structure, operation, or characteristic described in connection with such phrases can be included in at least one embodiment of the present technology. Thus, such phrases as used herein are not necessarily all referring to the same embodiment. Finally, it should be noted that various particular features, structures, operations, and characteristics of the embodiments described herein may be combined in any suitable manner in additional embodiments in accordance with the present technology.

I/we claim:

1. An implant for treating an aneurysm at a treatment location within a patient's vasculature at which a first blood vessel, a second blood vessel, and a third blood vessel converge, the implant comprising:
an elongate first body, wherein a length of the first body extends between a first end of the first body and an opposite second end of the first body, and wherein the first body comprises:
a first wall portion proximate to the first end, the first wall portion comprising a concave first inner surface and a convex first outer surface opposite the first inner surface along a thickness of the first wall portion,
a second wall portion proximate to the second end, the second wall portion comprising a concave second inner surface and a convex second outer surface opposite the second inner surface along a thickness of the second wall portion,
an arced third wall portion between the first and second wall portions, wherein a width of the third wall portion extends between a first side of the third wall portion and an opposite second side of the third wall portion,
a first side edge portion proximate to the first side, and
a second side edge portion proximate to the second side, wherein the second side edge portion is spaced apart from the first side edge portion to define an opening; and
a bulbous second body connected to the first body via the third wall portion, wherein a length of the second body extends laterally away from the first body, and
wherein the implant is transitionable between:
a deployed state in which:
the first wall portion engages a wall of the first blood vessel via the first outer surface,
the second wall portion engages a wall of the second blood vessel via the second outer surface,
the second body is disposed at least partially within the aneurysm, and
the opening is located relative to the third blood vessel such that at least some blood flow between the treatment location and the third blood vessel is via the opening, and
a delivery state in which profiles of the first body and the second body across their respective lengths are more compact than in the deployed state.

2. The implant of claim 1, wherein the first body comprises:
an arced first end edge portion proximate to the first end; and
an arced second end edge portion proximate to the second end.

3. The implant of claim 2, wherein the first and second side edge portions extend continuously from the first end edge portion to the second end edge portion.

4. The implant of claim 1, wherein:
the first body comprises:
an arced first end edge portion proximate to the first end, and
a looped second end edge portion proximate to the second end; and
the first wall portion is tubular.

5. The implant of claim 4, wherein the first and second side edge portions extend continuously from the third wall portion to the first end edge portion.

6. The implant of claim 1, wherein:
the first body comprises:
a looped first end edge portion proximate to the first end, and
a looped second end edge portion proximate to the second end; and
the first and second wall portions are tubular.

7. The implant of claim 1, wherein the implant is transitionable between the delivery state and an unconstrained state in which:
the second body extends away from the first body in a first direction;
the first wall portion extends away from the third wall portion in a second direction no more than 45 degrees offset from the first direction; and
the second wall portion extends away from the third wall portion in a third direction no more than 45 degrees offset from the first direction.

8. The implant of claim 1, wherein:
the third wall portion defines an arc along the width of the third wall portion between the first and second sides; and
an arc angle of the arc is no more than 180 degrees.

9. The implant of claim 1, wherein the second body comprises a concave inner surface and a convex outer surface opposite the inner surface of the second body along a thickness of the second body.

10. The implant of claim 1, wherein:
the implant defines an annular recess between the first and second bodies; and
the annular recess at least partially receives a neck of the aneurysm when the implant is in the deployed state.

11. The implant of claim 1, wherein:
the first and second bodies are connected to one another at a connection point along the length of the first body;
a first part of the length of the first body extends from the connection point to the first end;
a second part of the length of the first body extends from the connection point to the second end; and
the first part of the length of the first body is at least 30% shorter than the second part of the length of the first body to facilitate sequential deployment of the first and second wall portions at the treatment location.

12. The implant of claim 1, wherein:
the first and second bodies are connected to one another at a connection point along the length of the first body;
a first part of the length of the first body extends from the connection point to the first end;
a second part of the length of the first body extends from the connection point to the second end; and
the first part of the length of the first body is at least 3 millimeters shorter than the second part of the length of the first body to facilitate sequential deployment of the first and second wall portions at the treatment location.

13. The implant of claim 1, wherein:
the first, second, and third wall portions comprise a first mesh;
the second body comprises a second mesh; and
the implant further comprises a clamp at which the first and second meshes are connected to one another.

14. The implant of claim 1, wherein:
the second body comprises a wall comprising a concave fourth inner surface and a convex fourth outer surface opposite the fourth inner surface along a thickness of the wall; and
the second body engages a wall of the aneurysm via the fourth outer surface when the implant is in the deployed state.

15. A system for treating an aneurysm at a treatment location within a patient's vasculature, the system comprising:
a elongate shaft defining an axial lumen and comprising a proximal end portion and a distal end portion opposite the proximal end portion along a length of the shaft, wherein the shaft is configured to move the distal end portion intravascularly toward the treatment location; and
an implant in a low-profile delivery state within the lumen, wherein the implant is configured to expand from the delivery state to a deployed state, and wherein the implant comprises:
an elongate first body, wherein a length of the first body extends between a first end of the first body and an opposite second end of the first body, and
wherein the first body comprises:
a first wall portion proximate to the first end and extending along a first part of the length of the first body,
a second wall portion proximate to the second end and extending along a second part of the length of the first body, and
a third wall portion extending along a third part of the length of the first body between the first and second parts of the length of the first body, wherein the third wall portion is distal to the first and second wall portions along the length of the shaft, and
a second body connected to the first body via the third wall portion, wherein the second body is distal to the first body along the length of the shaft,
wherein a distance along the length of the shaft between the second body and the first end is at least 3 millimeters shorter than a distance along the length of the shaft between the second body and the second end to facilitate sequential deployment of the first and second wall portions at the treatment location.

16. The system of claim 15, wherein the implant is transitionable between the delivery state and an unconstrained state in which:
the second body extends away from the first body in a first direction;
the first wall portion extends away from the third wall portion in a second direction no more than 45 degrees offset from the first direction; and
the second wall portion extends longitudinally away from the third wall portion in a third direction no more than 45 degrees offset from the first direction.

17. A method for treating an aneurysm at a treatment location within a patient's vasculature at which a first blood vessel, a second blood vessel, and a third blood vessel converge, the method comprising:
locating a bulbous body of an implant within the aneurysm; and
locating an elongate body of the implant within the vasculature outside the aneurysm after locating the bulbous body, wherein locating the elongate body comprises:
engaging a wall of the first blood vessel via a convex first outer surface of a first wall portion of the elongate body,
engaging a wall of the second blood vessel via a convex second outer surface of a second wall portion of the elongate body,
engaging a wall of the vasculature between the first and second blood vessels via a convex third outer surface of a third wall portion of the elongate body, the third wall portion comprising a width extending between a first side of the third wall portion and an opposite second side of the third wall portion, wherein the elongate body comprises a first side edge portion proximate to the first side and a second side edge portion proximate to the second side, and wherein the second side edge portion is spaced apart from the first side edge portion to define an opening, and
locating the opening relative to the third blood vessel such that at least some blood flow between the treatment location and the third blood vessel is via the opening.

18. The method of claim 17, further comprising moving the implant intravascularly toward the treatment location while the implant is in a low-profile delivery state within an axial lumen of an elongate shaft, wherein locating the bulbous and elongate bodies of the implant comprises causing relative movement between the implant and the shaft to transition the implant from the delivery state toward an expanded deployed state.

19. The method of claim 18, wherein moving the implant intravascularly toward the treatment location comprises moving the implant intravascularly toward the treatment location while the third wall portion is distal to the first and second wall portions along a length of the shaft.

20. The method of claim 19, wherein:
a length of the elongate body extends between a first end of the elongate body and an opposite second end of the elongate body; and
moving the implant intravascularly toward the treatment location comprises moving the implant intravascularly toward the treatment location while a distance along the length of the shaft between the bulbous body and the first end is at least 30% shorter than a distance along the length of the shaft between the bulbous body and the second end.

21. The method of claim 17, wherein locating the elongate body comprises at least partially receiving a neck of the aneurysm at an annular recess defined by the implant between the bulbous body and the elongate body.
